(19) United States
(12) Patent Application Publication
(10) Pub. No.: US 2012/0282317 A1
(43) Pub. Date: Nov. 8, 2012

(54) Medicated popsicle system

(76) Inventor: Tamar Ekstein, New York, NY (US)

(21) Appl. No.: 13/068,252

(22) Filed: May 7, 2011

(51) Int. Cl. A61K 47/00; B29C 31/06

(52) U.S. Cl. 424/439; 425/130

(57) ABSTRACT

A system for providing medication in an edible frozen form consists of a popsicle mold, a popsicle holder with a core stick, handle and drip guard, and a liquid mixture containing sweeteners, edible colorants, and flavorings which is individually mixed with medication for each popsicle in order to add a custom flavoring to mask the taste of the medicine and encourage consumption by children. The system also includes a mixing implement and storage for the mixing implement incorporated into the popsicle mold, writable and erasable areas on the mold where information particularly relevant to the medicine type, dosage and administration schedule can be written and then changed as needed, an erasable writing implement, and storage for the writing implement incorporated into the popsicle mold.

### STATEMENT REGARDING FEDERALLY SPONSORED RESEARCH OR DEVELOPMENT

N/A

### BACKGROUND

The present invention relates generally to systems for making medicated popsicles in order to encourage children to consume prescribed or otherwise indicated medications.

Children may be reluctant to take required medication for a variety of reasons. The inherent taste of the medication may be displeasing, the flavorings used to conceal the inherent taste of the medication may be displeasing or have negative associations, the color may be displeasing, the mouth feel of the liquid may be displeasing, or the child may simply distrust anything being served on a spoon. Alternate forms in which to serve medication, particularly ones that children will have strongly positive associations with, are clearly of value.

Children generally like popsicles, which have the additional benefit of being soothing to sore throats. Medicated popsicles are an available retail item for a very limited number of medicines, and in a very limited number of flavors. Examples of these include freezer pops sold under the trademarks “Benylin” and “Pedialyte”. The former is available in only three flavors and the latter is available in only four flavors. These freezer pops also address only a narrow range of children's medicines. Further, a sick child may well be even more picky about what they'll eat than a healthy child, and the very limited number of flavors does not offer a sufficiently broad solution.

There are also numerous commercially available home-made popsicle kits with molds and popsicle sticks or holders, where fruit juice or other edible liquids may be poured in and frozen in a home freezer. However, these juices and liquids generally do not include the sort of concentrated flavorings which may be needed to sufficiently disguise the taste of particular medications, or the sort of bright colors which children may associate with popsicles as a festive treat. These kits also do not include any means for labeling directly on the popsicle mold what sort of medication is contained, the concentration, the flavoring, or when the mixture was prepared or served. Further, the size popsicles produced by these kits are significantly larger in volume than what is needed to provide a dose of medication plus sufficient amounts of colorants and flavorings.

There are several frozen medication mixture systems which bear discussion. For instance, U.S. Pub. No. 20080181934 entitled “Novel enhanced medicinal delivery system processes and products thereby especially useful for children” published Jun. 5, 2008, teaches the administering of medications particularly to children in the form of “gelatins and candies”. This publication also teaches the home mixing of medication with the modifying agents. The system further includes an embodiment wherein a dose of medication is combined with texturing, flavoring and sweetening agents and then frozen to be served as a medicated popsicle. However, there is an explicit requirement to include gel-type texturing agents thus complicating attempts at successful home formulation if additive concentrations are modified. Additionally, the publication does not teach anything about a suitable popsicle forming container, popsicle holder or means for recording data relevant to the medication dose.

Another example of a medicated popsicle is found in U.S. Pat. No. 6,162,468 entitled “Frozen aqueous solution with nutrients method of packaging and utilizing the same” filed Aug. 5, 1999 which teaches “a tasty means for taking a pre-measured dose of a medicine” which in all embodiments is pre-mixed. The storage and dispensing means taught is a “sealable plastic container” which is described as preferably a “releasably sealable long, slender plastic package” that is essentially a tubular bag made of flexible plastic film. The '468 patent does not teach a reusable container for freezing a popsicle, nor popsicle holder or means for recording data relevant to the medication dose, nor does it teach the home mixing of conventional store-bought medication with flavorings, wherein the caregiver and patient can select the specific mixture of flavorings.

Yet another example of a medicated popsicle is found in U.S. Pat. No. 5,431,915 entitled “Frozen oral medication delivery system and method” filed Dec. 20, 1993 which teaches several embodiments of an “oral medication delivery system for administering . . . in a frozen form” thus producing and administering a medicated popsicle. These embodiments include a pre-mixed flavored medication requiring shaking to improve even dispersion immediately prior to freezing, and two embodiments of a pre-measured two part dose of powdered medicine and liquid to provide flavoring and sweetening, the two parts being mixed immediately prior to freezing. For all embodiments the use of a sealable container is taught, with various methods of unsealing. The '915 patent does not teach a reusable container for freezing a popsicle, nor popsicle handle or means for recording data relevant to the medication dose, nor does it teach the home mixing of conventional store-bought medication with flavorings, wherein the caregiver and patient can select the specific mixture of flavorings.

A need exists, therefore, for a system and method of delivering doses of medication in frozen popsicle form wherein the flavor of the medication is masked by flavors more pleasing to children, the size of the popsicle is suitable for a child's dose of medication, the included popsicle handles are easy and convenient to use, and the system includes convenient means for recording information including medication type, dosage and time of administering.

### SUMMARY

In accordance with the present invention, a system for providing medication in an edible frozen form consists of a popsicle mold with at least one compartment for releasably holding a popsicle, a core stick for insertion into the compartment so that the popsicle may be frozen around the core stick, and a liquid mixture containing sweeteners, edible colorants, and flavorings which is individually mixed with medication for each popsicle in order to add a custom blend of edible colorants and flavorings to the medication.

Further, the system preferably includes a handle and drip guard attached to the core stick, a mixing implement consisting of a rod, paddle, whisk, spoon or a functional combination of such forms, a storage means for retaining the mixing implement incorporated into the popsicle mold, a writable and erasable area on the mold where information particularly relevant to the medicine type, dosage and administration schedule can be written, an erasable writing implement, and a storage means for retaining the writing implement incorporated into the popsicle mold.

### BRIEF DESCRIPTION OF THE SEVERAL VIEWS OF THE DRAWINGS

A more complete understanding of the invention and its advantages will be apparent from the detailed description taken in conjunction with the accompanying drawings in which:

FIG. 1 is an isometric view of a prior art popsicle mold;

FIG. 2 is an isometric view of a Medifreeze popsicle mold with writing implement and mixing implement;

FIG. 3 is an isometric view of a Medifreeze popsicle holder;

FIG. 4 is an isometric view of a Medifreeze popsicle mold with popsicle holders inserted; and

FIG. 5 is an isometric view of popsicle frozen material on the popsicle holder.

### DETAILED DESCRIPTION

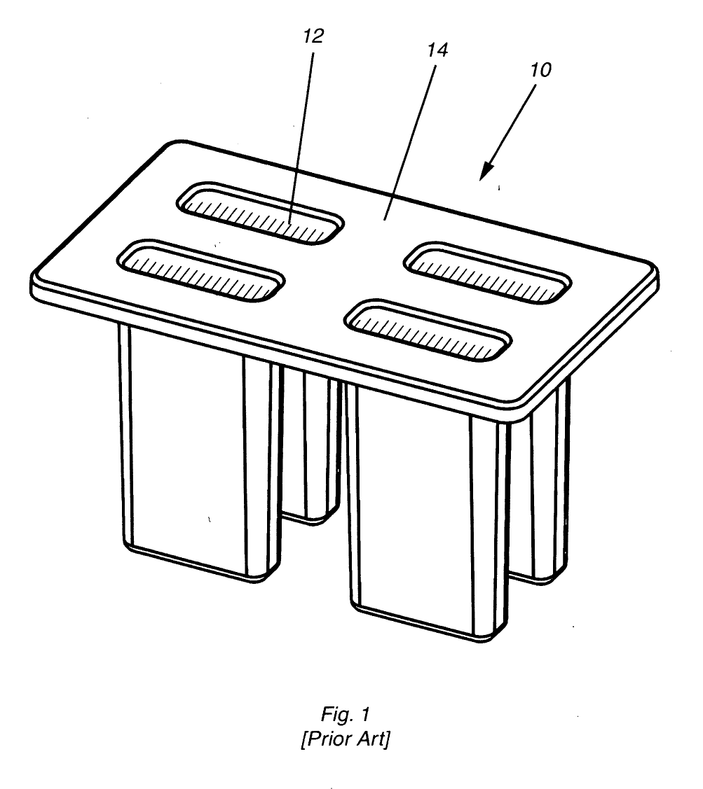

As shown in FIG. 1, a prior art popsicle mold 10 is composed primarily of a series of compartments 12 joined by a mold top surface 14. The compartments 12 as shown are rectangular in cross section but many other compartment shapes are well known in the art and are viewed as being functionally equivalent. The compartments are tapered to facilitate easy release of the popsicles and for ease in manufacturing. The compartments 12 are filled with an edible liquid mixture, a core stick is inserted to facilitate manipulation of the popsicle, and the mold is placed in a freezer to freeze the mixture and produce a popsicle. The core stick generally is part of a popsicle holder which in some home-use products includes a discrete handle and a drip guard which may also act to position or register the popsicle holder in relation to the compartment. Some home-use popsicle molds use other means to position a core stick in relation to the compartment, including a cover sheet with holes for the core sticks to protrude through. Generally the popsicle mold and popsicle holder are made of injection molded plastic, although some popsicle molds available for home use have at least some components made from metals such as food-grade aluminum or stainless steel.

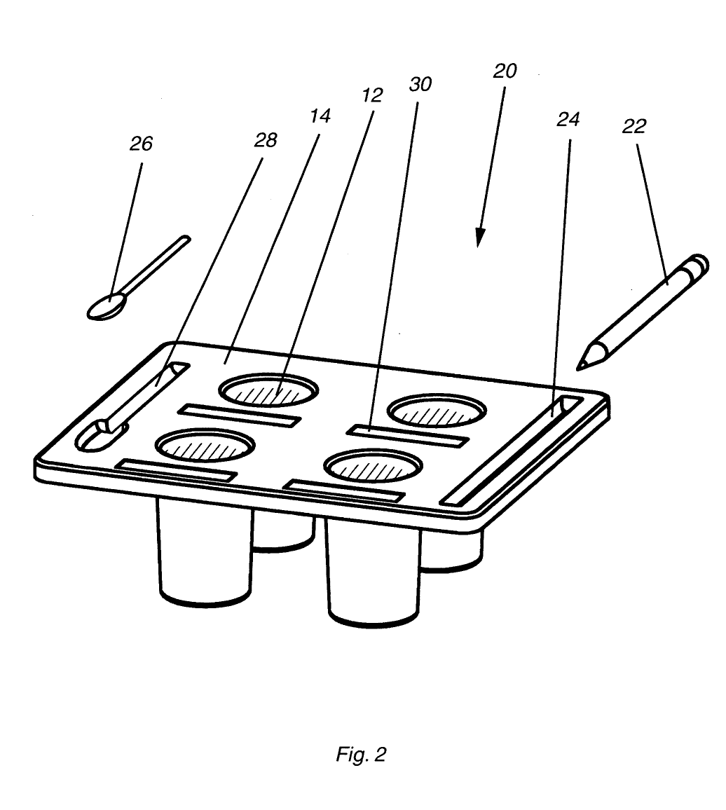

A Medifreeze popsicle mold 20 is shown in FIG. 2. As shown, there are preferably four compartments 12 since pediatric medication is generally administered no more than four times in one day. Each compartment 12 is approximately 2 inches in depth and approximately 1.25 inches in width at the opening, tapering inwards towards the bottom of the compartment to facilitate removal of the frozen popsicle. While the compartment 12 as shown is a tapered cylinder, any shape that is practical to manufacture that will produce a usable popsicle can be seen as within the scope of the invention. The preferred volume of each popsicle is approximately in the range of 30 mL to 40 mL, although a volume range from 25 mL to 60 mL is within the scope of the invention. Smaller volumes than this range are impractical for adequately masking the flavor of a medication dose, and larger volumes than this range will produce a popsicle sufficiently large that a small child may have difficulty finishing it quickly enough. Large popsicles run the risk of partial waste and loss due to excessive melting and possibly breaking apart. Each compartment 12 has an erasable writing area 30 where information such as medicine type, medicine dose, flavoring type, date of freezing, date of administering and time of administering can be written. Since the writing area is erasable, this information can be continuously modified with each successive use of the mold 20. Preferably, the erasable writing area 30 is formed directly into the body of the mold 20.

An erasable writing implement 22 is preferably included along with the mold 20, and a writing implement holder 24 or other mechanical feature for storing the writing implement 22 is also preferably incorporated into the body of the mold 20. As illustrated herein the writing implement holder 24 is formed into the top surface 14 of the mold 20. The erasable writing implement 22 may be any suitable type of pencil, pen, crayon, marker or any other variety of writing or marking device whose marks can be repeatedly removed after being applied to the erasable writing area 30.

A mixing implement 26 is preferably included along with the mold 20, allowing easy mixing of the medication and flavorings. The mixing implement 26 is preferably in the form of a spoon, although the form of a rod, paddle, whisk, spoon or any other form incorporating elements of one or more of these functional shapes is acceptable as a mixing implement 26. A mixing implement holder 28 or other mechanical feature for storing the mixing implement 26 is also preferably incorporated into the body of the mold 20. As illustrated herein the mixing implement holder 28 is formed into the top surface 14 of the mold 20.

The flavorings are preferably provided in premixed sealed packets, each containing a mixture of natural sweeteners, flavors and colors. Each flavor packet contains an individual dose for flavoring one popsicle. While a limited number of flavors may be provided in premixed form, in use the different premixed flavors may be combined in varying proportions to produce a far larger number of flavor combinations, thus further broadening the appeal of the medicated popsicles to children.

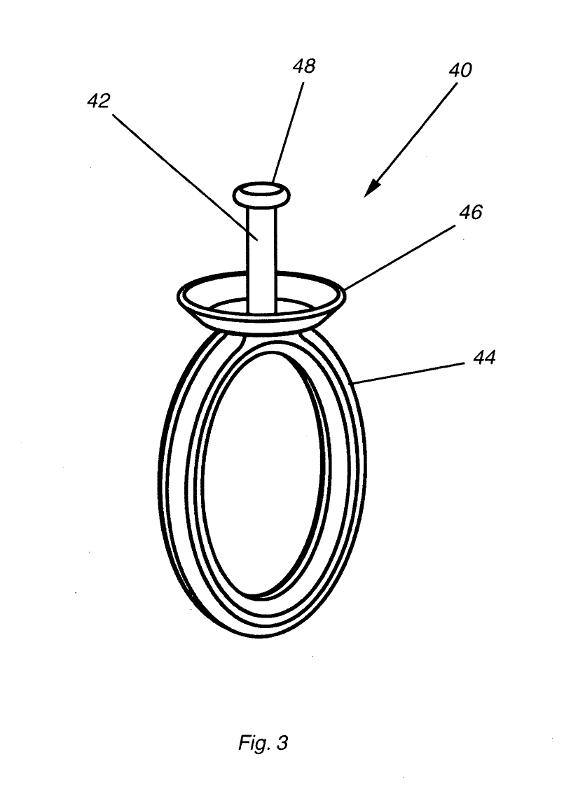

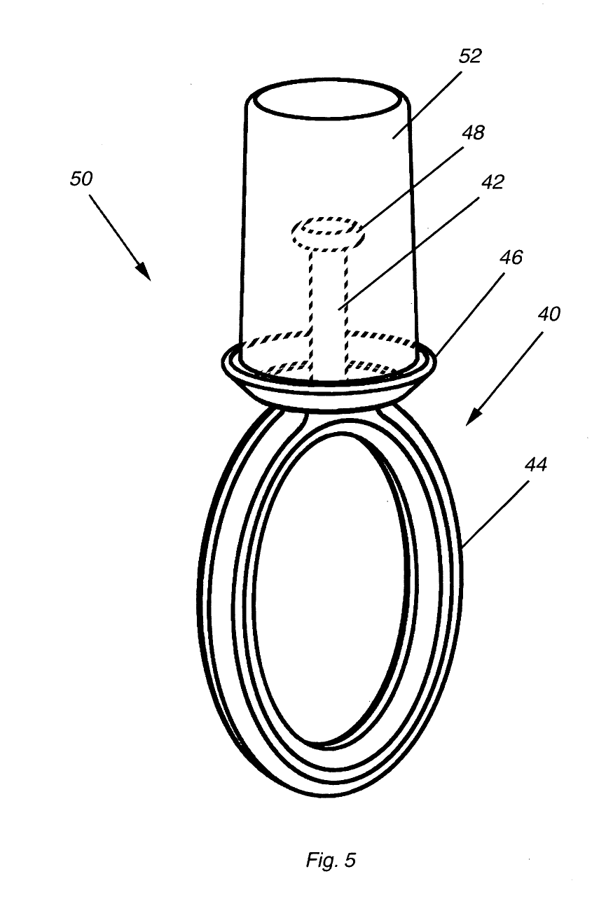

FIG. 3 shows a popsicle holder 40. FIG. 5 shows an entire popsicle 50 consisting of a popsicle holder 40 and frozen material 52. The popsicle holder 40 preferably comprises a handle 44, a core stick 42 with a retainer 48, and a drip guard 46. The core stick 42 and retainer 48 serve to anchor the frozen material 52 in place. The core stick 42 as shown extends approximately 1 inch into the frozen material, and the retainer is approximately centered within the volume of the frozen material. The handle 44 is formed as an open oval with internal dimensions preferably about 1.5 inches wide and 2.5 inches long to provide an easy grip for a child's hand. The drip guard 46 is preferably about 1.5 inches in diameter with an angled lip to retain any drips from the melting frozen material 52. If the compartment 12 is a shape other than round, the drip guard 46 is preferably of a shape to match that of the compartment 12. The retainer 48 may be any feature or features which produce a suitable mechanical interference between the core stick 42 and the frozen material 52, without interfering with the consumption of the frozen material 52.

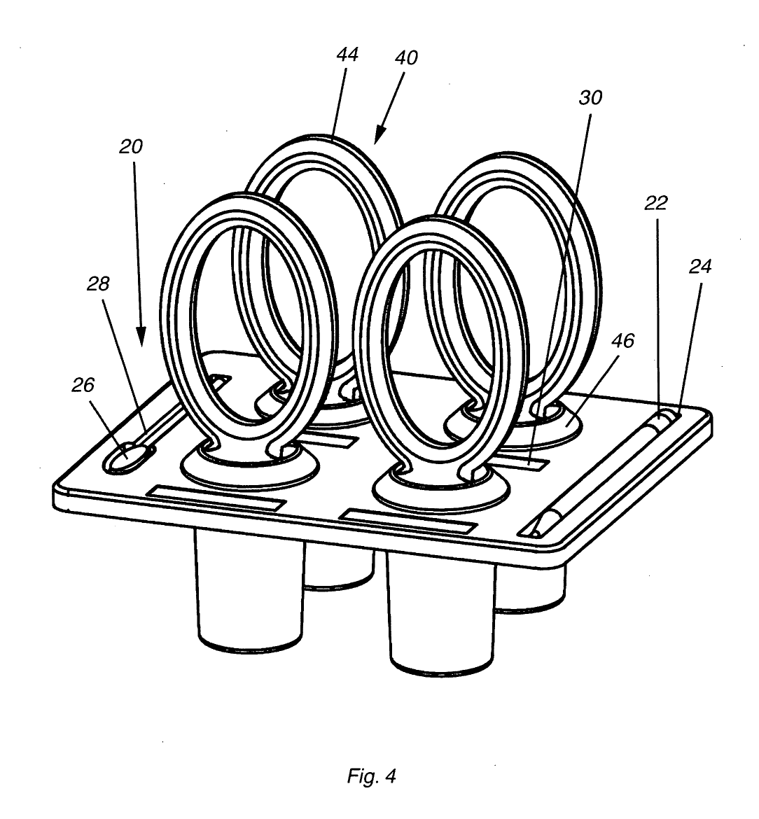

The entire Medifreeze popsicle system is shown in FIG. 4 with the popsicle holders 40 inserted into the compartments 12 in the mold 20. Preferably the system includes mechanical features to align the popsicle holders 40 with the compartments 12 in order to produce popsicles symmetrically formed around the core sticks 42. Such mechanical features are well known in the art and include but are not limited to ridges, recesses, walls, pins, slots and tabs.

The Medifreeze popsicle system is used as follows: A dose of medication is measured into one compartment 12. Up to four doses can be measured out at once, assuming all four compartments 12 are empty. The contents of a flavor packet is then poured into the compartment 12 and mixed with the medication using the included mixing implement 26. The mixing implement 26 is then cleaned and put back in the mixing implement holder 28. A popsicle holder 40 is placed on top of the compartment 12 with the core stick 42 submerged in the liquid mixture. The type and dose of medication are recorded on the erasable writing area 30 for that compartment 12 using the writing implement 22 which is then returned to the writing implement holder 24, and the mold 20 is placed in a freezer. This procedure can be followed for each empty compartment 12. When the liquid mixture is frozen and the popsicle 50 is ready to be administered, the time of administration is recorded on the erasable writing area 30. When the popsicle mold 20 is ready for cleaning and reuse, the written information is erased from the erasable writing areas 30.

Preferably the popsicle mold 20, the popsicle holder 40 and the mixing implement 26 are made from an inexpensive and readily available injection molded plastic such as polyethylene or polypropylene, although any injection moldable food-grade, freezer-safe polymer can be used. Preferably the erasable writing area 30 is formed by molding in texture to selected portions of the popsicle mold 20.

An alternate embodiment of the popsicle mold has removable compartments, thus permitting separate freezer storage of the popsicles without having to remove the popsicles from the mold compartments.

Another alternate embodiment of the popsicle mold has erasable writable areas on removable compartments.

Yet another alternate embodiment of the popsicle mold has erasable writable areas on the popsicle holders.

A further alternate embodiment of the popsicle mold and popsicle handle uses metal or other non-polymer materials for at least portions of the components so long as they are food-grade and freezer safe.

Still another alternate embodiment of the popsicle mold provides more or fewer than four compartments.

Yet another further alternate embodiment of the Medifreeze popsicle system uses replaceable erasable inserts for the writing areas.

An additional alternate embodiment of the Medifreeze popsicle system uses replaceable inserts for the writing areas.

Still another further alternate embodiment of the Medifreeze popsicle system uses an elongated core stick as the popsicle holder, without any discrete handle or any drip guard.

Still yet another further alternate embodiment of the Medifreeze popsicle system incorporates permanent text adjacent to or within the erasable writable areas, where this permanent text preferably includes data field titles such as medicine type, medicine dose, flavoring type, date of freezing, date of administering and time of administering.

Having described herein illustrative embodiments of the present invention, persons of ordinary skill in the art will appreciate various other features and advantages of the invention apart from those specifically described above. It should therefore be understood that the foregoing is only illustrative of the principles of the invention, and that various modifications and additions can be made by those skilled in the art without departing from the spirit and scope of the invention. Accordingly, the appended claims shall not be limited by the particular features that have been shown and described, but shall be construed also to cover any obvious modifications and equivalents thereof.

What is claimed is:

1. A system for providing medication in an edible frozen form, comprising:
a popsicle mold with at least one compartment for releasably holding a popsicle;
a popsicle holder for insertion into said compartment;
a flavoring mixture for adding to medication, said flavoring mixture containing ingredients from the group consisting of sweeteners, edible colorants, and flavorings;
said flavoring mixture being individually mixed with medication for said popsicle; and
whereby said flavoring mixture and medication being mixed and frozen in said compartment and around said popsicle holder produces a medicated frozen popsicle having individually selected flavors in order to mask the flavor of the medication and thus encourage consumption of the medication.

2. The system of claim 1, wherein said flavoring mixture is pre-mixed in sealed packets measured to flavor a single medicated popsicle.

3. The system of claim 1, wherein said flavoring mixture is in liquid form.

4. The system of claim 1, wherein said popsicle mold has a plurality of compartments and is of unitary molded plastic construction.

5. The system of claim 4, wherein said compartments are removable from said popsicle mold, thereby permitting said compartments containing said frozen popsicles to be transported separately or stored separately within a freezer.

6. The system of claim 1, wherein said compartment has a volume in the range of 25 mL to 60 mL.

7. The system of claim 1, wherein said compartment has a volume of approximately 30 mL to 40 mL.

8. The system of claim 1, wherein said popsicle holder comprises a handle attached to a core stick.

9. The system of claim 8, wherein said popsicle holder further comprises a drip guard providing means for catching melted drips falling off of said popsicle.

10. The system of claim 9, wherein said popsicle holder is of unitary molded plastic construction.

11. The system of claim 9, wherein said popsicle handle is a hollow generally oval shape with internal dimensions approximately 2.5 inches long by approximately 1.5 inches wide, thereby providing ease of grasping by a child's hand.

12. The system of claim 1, wherein said popsicle holder has a retainer portion to provide mechanical interference means for retaining material frozen around a portion of said popsicle holder.

13. The system of claim 12, wherein said retainer portion is an enlarged region at the distal end of said core stick, located approximately central to said frozen material.

14. The system of claim 1, further comprising means for positioning said popsicle holder relative to said compartment.

15. The system of claim 1, further comprising:
a mixing implement for mixing said medication with said flavoring mixture; and
storage means for retaining said mixing implement incorporated into said popsicle mold.

16. The system of claim 15, wherein said mixing implement has a functional shape incorporating features from the group consisting of rods, paddles, whisks and spoons.

17. The system of claim 1, further comprising a writable and erasable area on at least one portion of said mold whereby information can be repeatedly written and erased.

18. The system of claim 17, wherein said writable and erasable area is directly formed into at least one portion of said mold.

19. The system of claim 17, wherein the region adjacent to and including said writable and erasable area includes permanent text; and
said permanent text including titles from the group consisting of medicine type, medicine dose, flavoring type, date of freezing, date of administering and time of administering.

20. The system of claim 17, wherein said writable and erasable area is formed adjacent to said compartment.

21. The system of claim 17, wherein said writable and erasable area is directly formed into a portion of said popsicle holder.

22. The system of claim 17, wherein said writable and erasable area consists of a holder and a replaceable writable and erasable material.

23. The system of claim 17, wherein said writable and erasable area consists of a holder and a replaceable writable material.

24. The system of claim 17, further comprising storage means for retaining an erasable writing implement incorporated into said popsicle mold;

25. The system of claim 24, further comprising said erasable writing implement.

26. The system of claim 5, further comprising a writable and erasable area on a portion of each of said compartments whereby information can be repeatedly written and erased.

27. A system for providing medication in an edible frozen form, comprising:
a popsicle mold with at least one compartment for releasably holding a popsicle;
a popsicle holder having a core stick for insertion into said compartment, whereby said popsicle may be frozen around said core stick;
said popsicle holder having means for positioning within said compartment;
said popsicle holder having a handle and a drip guard;
a mixing implement having a functional shape including features from the group consisting of rods, paddles, whisks and spoons;
storage means for retaining said mixing implement incorporated into said popsicle mold;
a writable and erasable area on at least one portion of said mold whereby information can be repeatedly written and erased;
an erasable writing implement;
storage means for retaining said erasable writing implement incorporated into said popsicle mold;
a flavoring mixture containing ingredients from the group consisting of sweeteners, edible colorants, and flavorings;
said flavoring mixture being individually mixed with medication for each popsicle in order to add a custom flavoring to said medication; and,
whereby said flavoring mixture mixed with medication being frozen in said compartment and around said core stick portion of said popsicle holder produces a medicated frozen popsicle having individually selected flavoring in order to encourage consumption of said medication.

28. The system of claim 27, wherein said popsicle mold and said popsicle holder are made of injection molded polymer materials.

29. The system of claim 27, wherein said flavoring mixture is pre-mixed in sealed packets measured to flavor a single medicated popsicle.

30. A method for providing a medicated popsicle to a patient comprising the steps of:
measuring medication into a compartment of a popsicle forming mold;
measuring ingredients selected from the group consisting of sweeteners, edible colorants and flavorings into said compartment;
mixing said medication and said ingredients in said compartment;
inserting a core stick portion of a popsicle holder into said compartment;
placing said mold into a freezer whereby the mixture of said medication and said ingredients freeze into a custom flavored medicated popsicle around said core stick;
removing said medicated popsicle from said freezer; and
administering said medicated popsicle to said patient.

31. The method of claim 30, further comprising the step of:
noting information about said medicated popsicle on a writable and erasable area on said mold, said information selected from the group consisting of medication type, medication dose, flavoring type, date of freezing, date of administering and time of administering.

32. The method of claim 30, wherein said ingredients are the contents of a pre-mixed sealed packet measured to flavor a single medicated popsicle.

33. The method of claim 30, further comprising the steps of:
removing a mixing implement from a holder in said mold;
cleaning said mixing implement; and
replacing said mixing implement in said holder in said mold;

34. The method of claim 31, further comprising the steps of:
removing a writing implement from a writing implement holder in said mold; and
replacing said writing implement in said writing implement holder.
